(19) United States
(12) Patent Application Publication
(10) Pub. No.: US 2017/0007443 A1
(43) Pub. Date: Jan. 12, 2017

(54) CRYOGENIC DELIVERY SYSTEM

(71) Applicants: Mark Stephen Stroze, Wellington, FL (US); Brett Stanley Stroze, Wellington, FL (US)

(72) Inventors: Mark Stephen Stroze, Wellington, FL (US); Brett Stanley Stroze, Wellington, FL (US)

(21) Appl. No.: 15/165,356

(22) Filed: May 26, 2016

Related U.S. Application Data
(60) Provisional application No. 62/190,375, filed on Jul. 9, 2015.

(51) Int. Cl. A61F 7/00

(52) U.S. Cl. CPC A61F 7/0053; A61F 7/0085; A61F 2007/0064

(57) ABSTRACT

A cryogenic delivery and control system is provided. The cryogenic delivery and control system is supplied by an external cryogen supply reservoir, connected to a wet compartment which is in close communication to a man occupied dry compartment or chamber. The inhabited chamber of the present invention is used for cryogenically based, frigid atmosphere, on the order of about −100° to about −160° C., for the cooling and conditioning of living bodies.

### CROSS-REFERENCE TO RELATED APPLICATION

This application claims the benefit of priority of U.S. provisional application No. 62/190,375, filed Jul. 9, 2015, the contents of which are herein incorporated by reference.

### BACKGROUND OF THE INVENTION

The present invention relates generally to man inhabited chambers used for cryogenically based, frigid atmosphere cooling and conditioning of living bodies.

Past and current cryogen cryotherapy systems spray liquid cryogen against a plate inside the man-occupied chamber. The cryogenic spray-valve is cycled on and off in a bang-bang fashion. Natural convection is used to soak the user in frigid atmosphere. Common cryogenic liquid used is nitrogen or air. The user experiences non-beneficial wide temperature fluctuations when the cryogen liquid is sprayed against the deflector plate then turned off for a period of time. Current designs also exhibit a splattering of liquid droplets against the deflector plate located within the chamber, resulting in the user being burned by cryogen splatter droplets.

As can be seen, there is a need for an improved cryogenic therapy chambers.

### SUMMARY OF THE INVENTION

In one aspect of the present invention, a cryogenic delivery system comprises: a cryogen supply line; a compressed air tank; an air line fluidly connected to the compressed air tank; a cryogenic vessel plenum fluidly connected to the cryogen supply line and the air line; a chamber sized to fit an animal body within; an air plenum fluidly connected with the chamber; and a delivery line running from the cryogenic vessel to the air plenum, wherein the delivery line and the air plenum are operable to convert cryogen and air to a dry frigid gas and deliver the dry frigid gas to the chamber.

These and other features, aspects and advantages of the present invention will become better understood with reference to the following drawings, description and claims.

### BRIEF DESCRIPTION OF THE DRAWINGS

FIG. 1A is a side-rear perspective view of an embodiment of a cryogenic chamber system;

FIG. 1B is a front-side perspective view of the cryogenic delivery system of FIG. 1A;

FIG. 2 is a block diagram of the inside of the cryogenic delivery and control system of FIGS. 1A-1B; and

FIG. 3 is schematic view of the cryogenic delivery and control system of FIGS. 1A-1B.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

The present invention includes a multi-modal delivery of oxygenated cooled gas that is just above its liquid-vapor state that is continuous and may be directed at affected areas of a living body. The present invention includes economical delivery of industrial liquid cryogen converted to vapor to pre-chill an unoccupied chamber, and then delivery of medical-grade liquid cryogen converted to gas by mixing with blown air, delivered to the user when the chamber is occupied.

The present invention includes a more reliable and safer cryogen and control system by eliminating the possibility of cryogen splatter within the occupied chamber and with a more even and controllable temperature delivery which ensures a more beneficial user experience. Pneumatic conveyance using available atmospheric air is used to transform a cryogenic liquid to a gaseous state prior to the mixed stream entering the man-occupied chamber. Pneumatic delivery may be derived from stored air and/or an active conveyance system such as a compressor, blower or fan.

The present invention may use a positive or negative pneumatic pressure, relative to normal atmospheric pressure, induced in a cryogenic liquid container, upstream of the man-occupied chamber. A negative pressure may be induced in the cryogen vessel using a venturi based ejector blower, thereby mixing the cryogen with the flowing airstream. A positive pressure may be introduced into the cryogen vessel using a pressurized tube bubbling into the cryogen. In either case, the delta-pressure results in movement of and mixing of the cryogenic liquid and pressurized air.

A plurality of cryogenic vessels may be used to provide multiple sources of cooled air to the chamber. A conditioning circuit is used to pre-chill the delivery system and chamber prior to user entry. A separate cryogenic vessel is provided for the delivery of an acceptable grade (e.g. medical) of converted liquid to gas cryogen.

Pressurized pneumatic delivery and mixing has the added benefit of increasing the oxygen content of the cooled mixed gas delivered to the man-occupied chamber. Pressurized pneumatic delivery also allows for the conversion of a whole-body cryotherapy machine into a localized frigid gas delivery device. Phase transformation of liquid to gas is accomplished in the distribution assembly, prior to entry into the man-occupied chamber. Distribution assembly with a motive blower may also be used for the sublimational phase transformation of a commonly found frozen solid such as dry-ice.

Rectilinear interior of the chamber and distributed gas cryogen allows for easy expansion into multi-user configurations. Ducting may be built into each interior panel of the chamber for equal distribution of frigid gas to a multitude of coincident users.

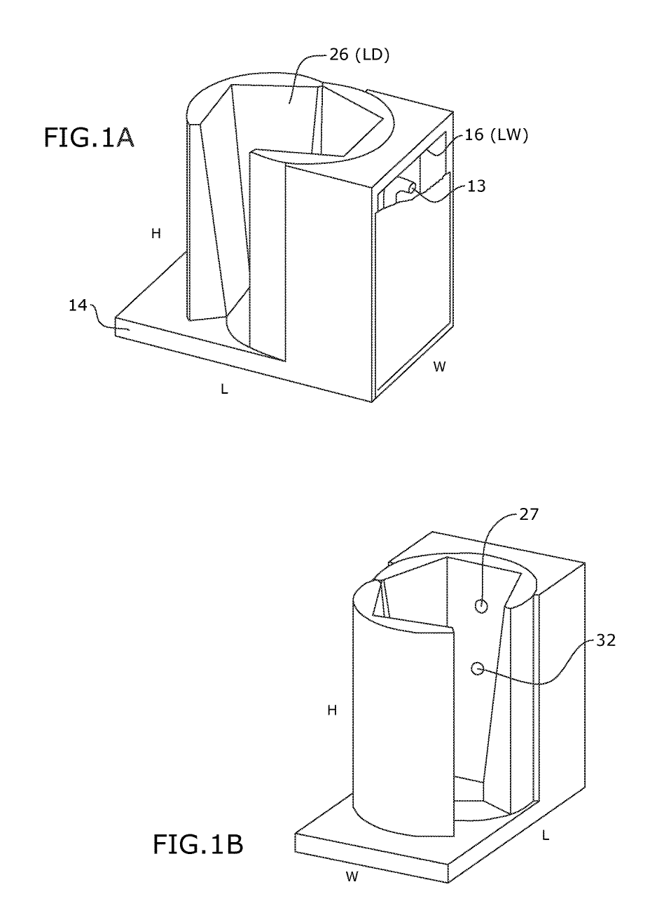

Referring to FIGS. 1 through 3, the present invention includes a cryogenic delivery and control system 14 supplied by an external cryogen supply reservoir 12, connected to a wet compartment 16 which is in close communication to a man occupied dry compartment 26 or chamber. The inhabited chamber of the present invention is used for cryogenically based, frigid atmosphere, on the order of about −100° to about −160° C., for the cooling and conditioning of living bodies.

Phase transformation of a cryogenic liquid to gas is performed in wet compartment 16 and frigid gas is delivered to dry compartment 26. Wet compartment 16 and dry compartment 26 are separate so that liquid cryogen cannot directly contact the user located in dry compartment 26. The wet compartment 16 may be open to the atmosphere to convert liquid into a vapor or sublimate a solid.

In certain embodiments, wet compartment 16 includes at least one cryogen supply line 13 or a plurality of cryogen supply lines (not shown) in communication with a control manifold 18 which distributes liquid cryogen and compressed air from compressed air tank 20 to an on-board open cryogenic vessel plenum 28. The compressed air tank 20 may include oxygen. Therefore, the cryogenic vessel plenum 28 may contain a blend of cryogen and oxygen.

Control manifold 18 is controlled via controller and computer 24. A first valve 15 is secured to the cryogen supply line 13. A second valve 15 secured to the air line 12. The valves 15 control the speed and amount of cryogen and the rate of air from the compressed air tank 20. The controller and computer 24 is operable to open and close the first valve 15 and the second valve 15. At least one sensor 27 is operable to deliver data to the controller and computer 24. The controller and computer 24 opens and closes the first valve 15 and the second valve 15 based on the data.

Cryogen liquid is transformed to vapor in open cryogenic vessel plenum 28 via pneumatic agitation and forced convective heat transfer. The liquid is sufficiently heated via agitation and mixing with relatively warm air to transform it into a gas prior to entering the man-occupied chamber. An additional safeguard is built into the system, whereby, the mixed gas flows upward before entrance into the man-occupied chamber. A dry frigid gas is produced in delivery line 29 and air plenum 30 as the result of continued heat transfer between cryogen and blown gas. Temperatures of other mixed-gas delivery locations may be further reduced by placing a liquid cryogen splatter shield in the chilled gas delivery line 29, whereby unmixed cryogen liquid is gravitationally returned to the cryogenic vessel plenum 28.

In certain embodiments, a startup thermal conditioning circuit is included, whereby, cryogen liquid line 17 supplies liquid directly to air plenum 30. Liquid is returned to open cryogenic vessel plenum 28 via liquid cryogen return line 31. The cryogen liquid line 17 may be used to condition the chamber to a moderately low temperature, such as −50° to about −75° C. prior to bringing the chamber down to −100° to about −160° C.

Dry compartment 26 delivers a dry frigid air/gas mixture from air plenum 30 to the user. The dry frigid gas may be delivered to specify body parts, such as lymphatic nodes under the arms and various contuded areas. The frigid air may include aromatherapy vapors and scents. Frigid air/gas mixture exhibits downward flow exits through return gas port 32 to be recirculated by blower 22. Blower 22 supplies recirculated dry chamber gases to open cryogenic vessel plenum 28. Dry compartment 26 is well insulated to reduce thermal transfer from the environment.

As mentioned above, blower assembly 22 transports frigid gas received from the return gas port 32 through its outlet to open cryogenic vessel plenum 28 when rotating. Blower assembly 22 can be a cylindrical blower, such as, for example, a rotary blower, a piston blower, a vane blower or another type of blower that is located in wet compartment 16. Blower motor found in the blower assembly 22 can be a brushless motor or any other type of electric motor that is able to power blower assembly 22 and is able to fit within wet compartment 16 of cryogenic delivery and control system 14.

Placement of air plenum 30 and outlet vents may be between the user's shoulders and knees. The return gas port 32 may be located in an area between the user's knees and feet. Therefore, the return gas port 32 may be below the air plenum within the chamber.

In certain embodiments, compressed air supply and storage 20, control manifold 18, cryogen blower 22 and controller and computer 24 can be mounted directly onto a platform (not shown) using bolts or other fasteners, but other methods of attachment depending on system requirements may also be used.

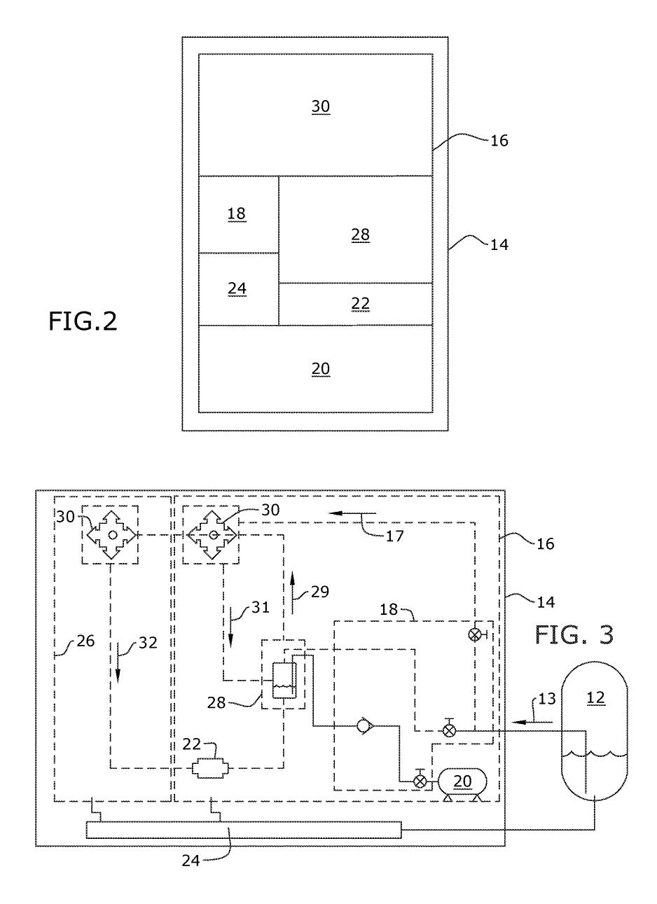

Though not shown in FIGS. 1A-2, cryogenic delivery and control system 14 can include a feed-through hole to bring electricity into housing to power controller and computer 24 and blower 22.

As mentioned above, the controller and computer 24 may be connected to a plurality of sensors 27 to monitor the user, dry chamber 26 and wet compartment 16 conditions. The controller and computer 24 may send signals for proportional motor drive to prevent introducing cryogen droplets into dry chamber 16. Controller and computer 24 can be connected to control and feedback elements contained within wet compartment 16 and dry chamber 26 through wires (through a feed-through in housing 14) or wirelessly to receive signals for controlling cryogenic delivery and control system 14. Controller and computer 24 then regulates the position of valves in manifold 18 based on those signals. Cryogenic delivery and control system 14 housing may include a flat base section and a cover section which extends in the depth dimension to contain the blowing system and the controller. The cryogenic and control system 14 housing may include dimensions having a width (W), length (L), height (H), length of wet compartment (LW) and length of dry compartment (LD). In the example shown, dimensions are a width W of about 48 inches (about 123 cm), height H of about 72 inches (about 183 cm), length of wet compartment LW of about 18 inches (about 61 cm) and length of dry compartment LD of about 42 inches (about 123 cm).

While the system has been shown with specific dimensions and specific placement of components, these are for example purposes only and may be varied. Additionally, cryogen blower and control system 14 could include other components, such as a shut-off valve depending on system requirements.

It should be understood, of course, that the foregoing relates to exemplary embodiments of the invention and that modifications may be made without departing from the spirit and scope of the invention as set forth in the following claims.

What is claimed is:

1. A cryogenic delivery system comprising:
a cryogen supply line;
a compressed air tank;
an air line fluidly connected to the compressed air tank;
a cryogenic vessel plenum fluidly connected to the cryogen supply line and the air line;
a chamber sized to fit an animal body within;
an air plenum fluidly connected with the chamber; and
a delivery line running from the cryogenic vessel to the air plenum, wherein the delivery line and the air plenum are operable to convert cryogen and air to a dry frigid gas and deliver the dry frigid gas to the chamber.

2. The cryogenic delivery system of claim 1, further comprising
a first valve secured to the cryogen supply line;
a second valve secured to the air line; and
a controller and computer operable to open and close the first valve and the second valve.

3. The cryogenic delivery system of claim 2, further comprising at least one sensor operable to deliver data to the controller and computer, wherein the controller and computer open and closes the first valve and the second valve based on the data.

4. The cryogenic delivery system of claim 1, further comprising an external cryogen supply reservoir fluidly connected to the cryogen supply line.

5. The cryogenic delivery system of claim 1, further comprising a cryogen liquid line running from the cryogen supply line to the air plenum.

6. The cryogenic delivery system of claim 5, further comprising a liquid cryogen return line running from the air plenum to the cryogenic vessel plenum.

7. The cryogenic delivery system of claim 1, further comprising:
a blower;
a return gas port fluidly connecting the chamber and the cryogenic vessel plenum, wherein
the blower is operable to pump the dry frigid gas out of the chamber and back into the cryogenic vessel plenum.

8. The cryogenic delivery system of claim 7, wherein the return gas port is disposed below the air plenum within the chamber.

9. The cryogenic delivery system of claim 1, wherein the chamber is insulated.

10. The cryogenic delivery system of claim 1, wherein the chamber is sized to hit a human body within.
